(19) United States
(12) Patent Application Publication
(10) Pub. No.: US 2023/0134196 A1
(43) Pub. Date: May 4, 2023

(54) CIRCUMFERENTIALLY WRAPPABLE ELECTRODE FOR USE WITH METAL SURGICAL IMPLANTS

(71) Applicant: Garwood Medical Devices, LLC, Buffalo, NY (US)

(72) Inventor: Jackson G. Hobble, Buffalo, NY (US)

(21) Appl. No.: 17/802,709

(22) PCT Filed: Feb. 18, 2021
(86) PCT No.: PCT/US2021/010004

§ 371 Date: Aug. 26, 2022

Related U.S. Application Data
(60) Provisional application No. 62/984,332, filed on Mar. 3, 2020.

(51) Int. Cl. A61N 1/20; A61N 1/05; B32B 5/18; B32B 9/00; B32B 9/04; B32B 15/20; B32B 15/02; B32B 3/08; B32B 3/26

(52) U.S. Cl. CPC A61N 1/205; A61N 1/05; B32B 5/18; B32B 9/007; B32B 9/046; B32B 15/20; B32B 9/041; B32B 15/02; B32B 3/08; B32B 3/266; A61F 2/38

(57) ABSTRACT

A wrappable electrode includes a flexible covering and a lead wire connecting the electrode to a stimulating device. The wrappable electrode further includes an adhesive layer to enable attachment to the skin of a patient and an inert conductive layer to which the lead wire is electrically coupled. The electrode is sized to be wrapped about at least a majority of a circumference of a limb of a patient in proximity to a metal surgically implanted device. The adhesive layer includes a buffered hydrogel. The electrode includes a separate conductive layer to evenly distribute electrical current relative to the metal implanted device, with the electrode serving as an anode and the implanted device serving as a cathode in a CVCES treatment system. The electrode can further include at least one feature to ensure proper placement on the skin of the patient.

### CROSS REFERENCE TO RELATED APPLICATION

This application is a national stage application under 35 U.S.C. § 371 of International Application No. PCT/US2021/010004, filed Feb. 18, 2021, which claims priority under applicable portions of 35 U.S.C. § 119 to U.S. Patent Application Ser. No. 62/984,332, filed Mar. 3, 2020, the entire contents of each application being herein incorporated by reference herein.

### TECHNICAL FIELD

This application is generally related to the field of surgically implanted orthopedic devices, and more specifically to a wrappable electrode used as part of a system to remove biofilm from a metal implant.

### BACKGROUND

Metal implants are orthopedic devices or apparatus that are used in patients with many different injuries or medical problems. In particular, metal implants may be used for any individual that needs to replace joints. For example, a metal implant may be used to replace a patient's hips or knees. One potential problem with metal implants is that they tend to allow for the growth of bacteria on the surface. This may increase the patient's risk for an infection, which could require the potential removal of the implant. To decrease the risk of infection, electrodes can provide electrical stimulation to disrupt the growth of bacteria.

It has been shown in scientific literature that application of cathodic current to metal samples create chemical reactions at that surface that can disrupt and kill bacterial biofilms that exist on the metal. For electrochemical processes to occur, there must be an anode and a cathode within an electrolyte solution. The anode is a metallic surface where oxidative reactions occur, and the cathode is another metallic surface where reduction reactions occur. A reduction reaction is essentially when the material of interest gains electrons and thereby decreases the oxidation state of the molecules. The electrolyte that the anode and cathode each reside in provides the electrical connection by facilitating the flow of electrons shuttled by ion carriers, such as sodium or potassium ions. Electrons are driven from the anode to the cathode through the electrical path via a potentiostat.

A potentiostat is a stimulating device or instrument used to drive electrical current from a counter electrode to a working electrode in order to keep the voltage on the working electrode at a constant value, as compared to a stable reference electrode. In the case of Cathodic Voltage Controlled Electrical Stimulation (or CVCES), the anode represents the counter electrode and the cathode represents the working electrode. Using a potentiostat, a user can dictate which electrochemical process is actually occurring on the working electrode and at what rate it occurs simply by adjusting the applied voltage parameters. The counter electrode has specific physical, electrical, and chemical requirements it must meet in order to sufficiently facilitate CVCES, especially in a clinical environment in which a patient's health is concerned.

The CVCES technique in a clinical setting has been shown as a way to fight bacterial biofilm infections on metallic implants in the most minimally invasive way possible. In this setting, the patient's body provides the electrolytic solution and therefore acts as an electrochemical cell by using the metal implant (working electrode) as the cathode and the counter electrode as the anode.

There is a general need in the field to improve the reliability and consistency of the above-described treatment systems. Moreover, issues involving the efficacy of the above described technique have been impacted by certain limitations in the design of the skin applied counter electrode.

### BRIEF DESCRIPTION

The disclosed invention presents a novel way of increasing the efficiency of the treatment of metal implants, while maintaining patient safety parameters and its minimally invasive profile.

Therefore and according to one aspect, there is provided a wrappable electrode configured for use in an electrochemical system for treating a metal surgically implanted device, the electrode comprising a flexible covering, a lead wire configured for connecting the electrode to a stimulating device, an adhesive layer to enable the electrode to be attached to the skin of a patient, and an inert conductive layer to which the lead wire is electrically connected. The electrode, including each of the resident layers, is sized and configured to be wrapped circumferentially about the limb of a patient so as to uniformly and evenly distribute treatment onto a surface of the implanted device. According to a preferred embodiment, the electrode is substantially wrapped entirely about an entire limb of a patient (arm, leg) having the implanted device, although the electrode can provide improved treatment results by covering at least 50 percent of the limb of the patient.

According to at least one version, the electrode further comprises another conductive layer disposed between the inert conductive layer and the flexible covering. In at least one embodiment, the latter conductive layer is made from copper, which can be provided, for example, as a mesh or a solid sheet of material. This conductive layer enables better effective electrification upon the inert (anodic) conductive layer of the electrode.

The adhesive layer of the electrode can comprise a hydrogel. According to a preferred version, the hydrogel further comprises a buffering compound.

According to at least one embodiment, the electrode further comprises at least one feature disposed on the flexible covering configured to align the electrode relative to the patient. In one version, the at least one feature aligns the electrode with a known anatomical landmark of the patient.

The metal surgically implanted device is preferably a working electrode with the herein described electrode acting as a counter electrode of the electrochemical treatment system, each couplable to the stimulating device that applies an electrical current between the working and counter electrodes. In a CVVES system, the herein described electrode serves as the anode for the electrochemical reaction and the implant acts as the cathode, enabling biofilm to be removed under the application of a suitable voltage.

According to at least one embodiment, the herein described electrode can further comprise at least one feature for positioning a separate but proximate reference electrode. The at least one positioning feature comprises a cut-out area formed on the electrode, the cut-out area being sized and configured for placement of the reference electrode.

According to another aspect of the invention, there is disposed a system configured for treatment of a metallic surgically implanted device, the system comprising a stimulating device capable of providing an electrical current, and an electrode configured for attachment to the skin of a patient in proximity to the metallic surgically implanted device, each of the metallic surgically implanted device and the electrode being electrically coupled to the stimulating device in which the metallic surgically implanted device is a working electrode and the electrode is a counter electrode of a formed electrochemical cell, and in which the electrode is sized and configured to be wrapped circumferentially about a limb of the patient in order to uniformly and evenly provide treatment on a surface of the impanted device. In a preferred embodiment, the electrode is configured to be entirely wrapped about the limb (arm, leg), but minimally the electrode can be wrapped at least 50 percent about the limb to provide improved results.

In at least one embodiment, the electrode comprises a flexible covering, a lead wire configured for connecting the electrode to the stimulating device of the system, an adhesive layer to enable the electrode to be attached to the skin of a patient, and an inert conductive layer to which the lead wire is electrically attached.

The electrode can further comprise another conductive layer disposed between the inert conductive layer and the flexible covering. According to at least one preferred embodiment, the latter conductive layer is made from copper and can be provided as a mesh or a sheet in which the conductive layer enables more uniform electrification of the inert (anodic) conductive layer of the electrode.

The adhesive layer of the electrode can comprise a hydrogel and according to a preferred embodiment, the hydrogel further comprises a buffering compound.

In at least one version, the electrode further comprises at least one feature disposed on the flexible covering that is configured to align the electrode relative to the patient. In at least one preferred embodiment, the at least one feature repeatably aligns the electrode relative to a known anatomical landmark. In another version, the herein described electrode further comprises at least one feature for positioning a separate but proximate reference electrode. The at least one positioning feature can comprise at least one cut-out area formed on the counter electrode, the cut-out area being sized and configured for placement of the reference electrode.

The inert conductive layer can be made from carbon. When disposed in relation to the metallic surgically implanted device, the inert conductive layer serves as the anode of the electrochemical reaction wherein suitable current is provided by the stimulating device through the coupled lead wire to the electrode.

This invention is based on a system in which DC electrical current is applied to a surgically implanted device, such as a knee replacement, in order to electrochemically clear and disrupt harmful bacterial biofilm from the metallic surface. The system requires at least two electrodes to effectively transfer the DC electrical current to the metal implant. One electrode is the implant itself, referred to as the working electrode, which is connected to the stimulating device by mechanical means, such as a needle or other subdermal attachment. The second electrode, referred to as the working electrode, is adhered to the skin of the patient.

The invention as disclosed is a novel design of the skin-based counter electrode that addresses and improves several problems with the overall system of treatment. One significant feature of the design is that the electrode wraps around the full periphery of the limb. This feature inherently does two (2) things to improve the overall treatment. First and if the skin electrode was a common patch located on one side of the implant, the natural tendency of the electrochemical reaction would become more intense on the side of the implant that is closest to the implant, thereby creating an uneven treatment on the implant. Using the herein described electrode that preferably covers the entire circumference or at least 50 percent of the limb of the patient in proximity to the implant provides an equidistant or substantially equidistant path for electrical current to every point on the implant, thereby creating an even and uniform distribution of treatment upon a surface of the metal implant. Second and if the skin electrode was only on one side of the limb, all of the electrical current directed to the implant would enter the body at a smaller, single area of the skin and tissue. Depending on the amount of electrical current entering the body of the patient, concentrating the current locally all at one spot could potentially cause chemical burns or thermal necrosis to the localized tissue. Using the herein described electrode, however, that covers substantially a majority or more preferably the entire periphery of the limb (i.e., arm, leg) of the patient allows the electrical current entering the body to become much more widely distributed.

Typically, electrodes are electrically attached via a single point of contact to the lead wire. With an electrode of this size that covers a larger overall area of the limb of the patient, a single point of contact may require the stimulating device providing the electrical potential to generate larger voltages. To keep the voltage requirement as low as possible and according to a preferred embodiment, an additional conductive layer made preferably from a copper mesh or suitable matrix is embedded behind a chemically inert (carbon) conductive surface layer of the electrode to more evenly spread the point of contact over a larger area. In some instances, these treatments employed on a surgical implant may further employ a third electrode, referred to as a reference electrode, which is used in conjunction with the working and counter electrodes. In such instances, it is important that the reference electrode be placed in a consistent anatomical location from patient to patient. If the reference electrode is inconsistently positioned or disposed on the body of the patient, resistance measures between the metal implant and the reference electrode can change which may alter the rate of the reaction in certain situations. Therefore and according to at least one version, the counter electrode further includes at least one feature that is configured to align with anatomical landmarks of the body, such as the patella. These alignment feature(s) provide consistent placement zones for other electrodes in the system in order to provide more consistent treatment in terms of current draw.

Advantageously, this invention is unique because it improves treatment outcomes and consistency for a medical procedures relative to surgically implanted devices, whose purpose is to eliminate biofilm infections of the devices.

The disclosed invention provides a means to promote an evenly distributed electrical current upon a metal implant, such as the femoral or tibial components of a knee implant to increase treatment consistency and effectiveness. The invention also distributes the anodic current on the skin electrode over a larger area, as compared to other electrode configurations, thereby decreasing the likelihood of harm or injury to the skin or bodily tissue beneath the electrode. The herein described electrode further resolves a need to optimize voltage capabilities of the stimulating device, such as a potentiostat or similar means, through the use of conductive meshes adhered behind the electrode's conductive surface. In addition and according to at least one version, the herein described electrode provides consistent placement zones for other electrodes in the electrochemical system to provide more consistent treatment in terms of current draw.

### BRIEF DESCRIPTION OF THE DRAWINGS

These and other features and advantages will be readily apparent from the following Detailed Description, which should be read in conjunction with the accompanying drawings.

FIG. 1 depicts a schematic view of an electrochemical system including a known electrode in conjunction with a surgically implanted device (a metal implant);

FIG. 2 depicts a schematic view of an electrochemical system that includes an electrode, which is made in accordance with aspects of the invention;

FIG. 3 is a diagrammatic view of an electrode made in accordance with aspects of the invention, as attached to an electrochemical system for treating a metal implant; and

FIG. 4 is a partially exploded view of a portion of an electrode made in accordance with aspects of the invention.

### DETAILED DESCRIPTION

The following relates to embodiments directed to an improved and novel electrode design for use in electrochemical systems and processes for treatment of metallic surgically implanted devices (hereinafter also synonymously referred to throughout as “metal implants” or “metallic implants”). As discussed herein, the embodiments that are described are to a specific surgically implanted device (knee replacement). However, it will be understood that the concepts described are applicable to literally any metallic surgically implanted orthopedic device. In addition, a number of terms are used throughout this description in order to provide a suitable frame of reference for the accompanying drawings. These terms, which may include “front”, “rear”, “interior”, exterior”, “distal”, “proximal”, “top”, “bottom”, “upper” and “lower”, and the like are not intended to overly limit the intended scope of the invention, except where so specifically indicated.

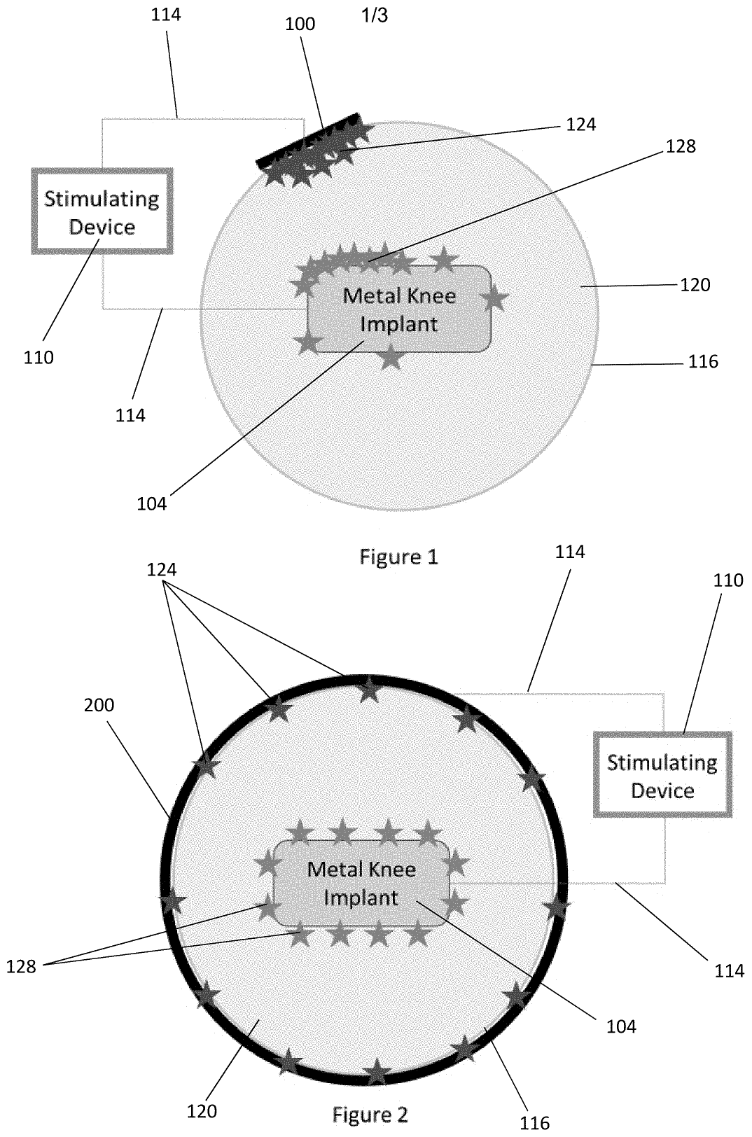

FIG. 1 depicts a scenario in which a traditional patch (TENS) electrode 100 is placed on one side of the skin 116 of a patient with a small surface area (represented by the short black line) as part of an electrochemical system used to provide treatment of a surgically implanted device and more specifically a metal implant 104. The patch electrode 100 is defined by a local profile (typically about 2″ by 2″) having an adhesive layer, an internal conductive layer as well as an outer covering layer. The known patch electrode 100 may include a hydrogel layer as part of the adhesive layer or in some instances be defined by an ultrasound gel beneath a dry carbon layer. Such known electrodes 100 that are typically configured for muscle stimulation.

In this instance, the metal implant 104 is a knee replacement having femoral and tibial components. In the depicted electrochemical treatment system, the metal implant 104 is electrically coupled to a stimulating device 110, such as a potentiostat, which is capable of applying electrical current, using needles or other connection means. The electrical connections between the stimulating device 110 and the metal implant 104 are each diagrammatically shown by lines 114, thereby forming a working electrode (cathode) at the metal implant 104. The number of needles or other connection means attachable between the metal implant 104 and the stimulating device 110 can be suitably varied.

As shown in FIG. 1, electrical connections are also made between the stimulating device 110 and the skin electrode via a lead wire, which in this instance is also diagrammatically shown as 114. The patch electrode 100 is attached adhesively to the skin of the patient on one local side of the leg of the patient and further to the stimulating device 110, and forms a counter electrode (anode) of the treatment system with the patch electrode 100 and metal implant 104 (cathode) forming an electrochemical cell.

FIG. 1 represents an idealized cross-sectional view of a leg at the knee where the metal implant 104 is in the center of the joint. The circular region 116 represents the skin, with the shading 120 within the circle 116 representing the flesh, the stars 124 representing anodic current flow, and the stars 128 representing cathodic current flow as applied by the stimulating device 110. It can be seen in this scenario that the stars 128 are clustered heavily on the surface of the metal implant 104 closest to the skin electrode 100, with very few stars 128 on the further sides of the metal implant 104. The distance between the skin electrode 100 and the opposite side of the metal implant 104 increases the natural resistance between the two electrode zones, and thus less electrical current will flow there. That is to say, there is a correlation between anode-to-cathode distance and resistance. Resistance will also change depending on other factors in the system such as muscle composition, fat composition, bone, skin hydration, and overall body hydration. Ultimately, this causes the side of the metal implant 104 with high densities of cathodic reactions to receive a disproportionate amount of treatment, as compared to the low-density reactive side, creating a clinical treatment that is far from ideal.

FIG. 2 depicts the same leg as in FIG. 1, using the same electrochemically-based treatment system. Similar features are labeled with the same reference numbers, including the same symbols as those previously discussed in FIG. 1, for the sake of clarity. Both of the stars 124, 128 representing anodic and cathodic current flow, respectively, are in the same quantity in both FIGS. 1 and 2. In this version, the stimulating device 110 is electrically coupled via conductive needles or similar means 114 to the metal implant 104, the latter acting as a working electrode of the created electrochemical cell with the counter electrode 200 being attached to the skin of the patient. In this version, the counter electrode 200 is sized and configured to be wrapped entirely about the leg of the patient, and more specifically around the entire knee joint peripherally. As can be seen diagrammatically in FIG. 2, the counter electrode 200 normalizes the distance between itself and any singular point on the metal implant 104, at least to a much higher degree than in the prior art example of FIG. 1, which therefore creates a near equivalent resistance path from all points on the surface of the metal implant 104. Because of this, the stars 124, 128 that represent the electrochemical reaction become homogeneously dense upon both the surface of the metal implant 104 and the counter electrode 200. Providing an electrode disposed in a wrappable manner therefore provides a more consistent and predictable treatment and disrupts bacterial biofilm found on the implant 104. Consistent treatment is especially important when treating biofilms because, if portions of the biofilm do not receive adequate treatment, as would be the case in FIG. 1, the biofilm is liable to simply regrow and continue to cause infectious issues for the patient. In summary, the electrode 200 described herein is designed in a novel way that covers the full periphery of the limb, thereby providing an equidistant electrical current path to every point on the metal implant 104, and creating an even distribution of treatment thereupon.

As noted, a novel feature of the present electrode design is that the counter electrode 200 wraps around at least a majority or more preferably the full circumference of the limb in proximity relative to the metal implant. This feature inherently does two (2) things to improve the treatment of the metal implant 104. First and if the electrode was a common patch located on one side of the implant 104, such as the traditional patch electrode 100, FIG. 1, the natural tendency of the electrochemical reaction will become more intense on the side of the metal implant 104 that is closest to the patch electrode 100, thereby creating an uneven treatment on the implant 104. This behavior occurs because the electrical current applied by the stimulating device 110 will naturally want to flow through the path of the least resistance to the other electrode.

A second advantageous improvement that the herein described electrode 200, FIG. 2, demonstrates over traditional patch electrodes 100 is shown by side by side examination of FIGS. 1 and 2. In FIG. 1, the behavior of the stars 124 only, i.e., the anodic reaction, is observed to be extremely dense on the skin electrode 100 because that is the only area in which the anodic reaction can occur. If the skin electrode 100 is installed in this configuration, all of the electrical current interacting with the skin electrode 100 would enter the body in a smaller, singular area of the skin and tissue. Depending on the amount of current entering the body, concentrating it all to one spot could cause severe chemical burns or thermal necrosis to the localized tissue, especially the skin. High current density causes damage to the skin because each individual chemical reaction causes the local pH to increase or decrease (depending on if the reaction is anodic or cathodic). Because the skin electrode 100 represents the anodic reaction, the pH will become more acidic. As the reactions become denser over a certain area of electrode, the acid being created will become more concentrated and the pH will decrease faster. If a low pH is concentrated enough and in contact with the skin for long enough, the skin will experience chemical burns. Using the electrode 200, FIG. 2, that covers the full circumference or at least a majority of the circumference of the limb allows the electrical current entering the body to become much more widely distributed over the surface of the electrode/skin, subsequently lowering the anodic current density, slowing down the decrease in pH per unit of area, and creating a safer treatment for the patient.

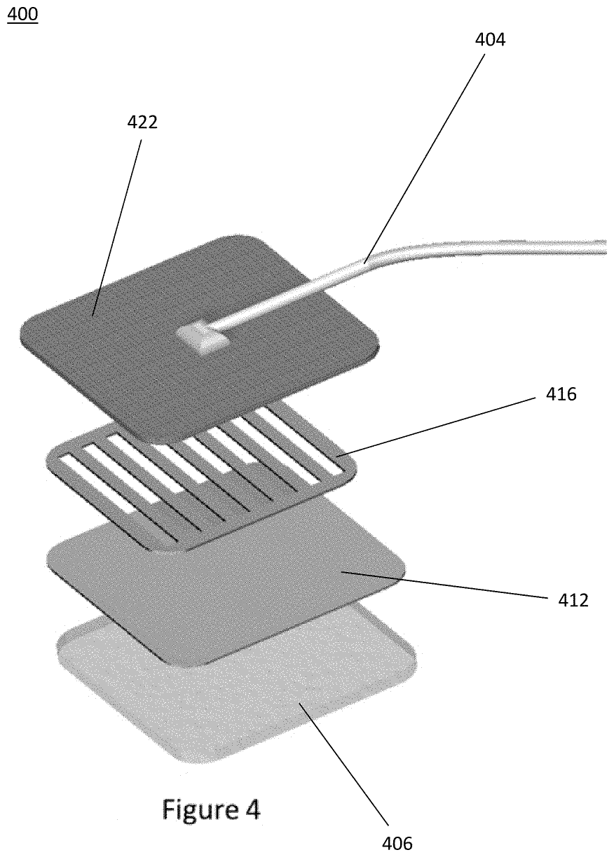

Further specifics of a novel electrode design in accordance with an exemplary embodiment are further discussed with brief reference to FIG. 4, depicting a sectioned view of a portion of an electrode 400. This electrode 400 is only partially shown to show the internal construction but is intended to be defined by a wrappable configuration similar to the electrode 200 of FIG. 2 that is sized and configured to be wrapped over a majority (greater than 50 percent) or more preferably the entire circumference of a limb of a patient in proximity to an implanted orthopedic device. In brief and as detailed in greater detail below, the general components of the novel electrode 400 according to this embodiment include a lead wire 404 that extends from a stimulating device such as potentiostat 110, FIGS. 1, 2, wherein the electrode 400 is fabricated with a series of stacked layers that include an adhesive exterior layer 422, made preferably from a fabric, a conductive layer 416, a conductive anodic film or surface layer 412, and a buffered hydrogel 406, in addition to the preferred sizing of the electrode 400 to permit wrapping as previously discussed. Details relating to the operation of the potentiostat 110 are known to those in the field and do not require further explanation as to their use in CVCES treatment systems. Each of the foregoing features, in addition to at least one feature for aligning/locating the electrode on the skin of the patient, are now described in greater detail.

One feature of the novel electrode 400 is to help improve patient safety is a buffering system within an adhesive hydrogel that acts to neutralize any acidic pH changes at the electrode to skin interface. A hydrogel is a solid gel that is composed of a fibrous mesh and water. Hydrogels have a large water content and are typically both conductive and adhesive. Nearly all stimulating and monitoring electrodes on the market use hydrogels as the adhesive, as well as the conductive interface between the conductive electrode surface and the skin of the patient. In the case of using electrodes to stimulate metal implants in order to treat a bacterial biofilm, the hydrogel is the first electrolyte environment that the counter electrode interfaces with to create the electrochemical reaction of converting electrons in a chemical state to a purely conductive state. That means that the hydrogel will be the first layer that turns acidic from the anodic reaction. Once the hydrogel becomes acidic enough, the hydrogel will start to chemically burn the skin of the patient. Therefore and according to an aspect of the herein described electrode, the hydrogel is preferably infused with a chemical buffer that counteracts changes in local pH and incorporated in a layer 406, FIG. 4, of the electrode 400, FIG. 4. When hydrogen ions are produced (the bases of acid), the buffering compound(s) in the hydrogel that are present bind with the hydrogen ions and neutralize them. Accordingly, the pH cannot begin to decrease until the buffering capacity of the hydrogel is first depleted, thus adding yet another layer of patient safety while undergoing treatment to remove biofilm from the metal implant. In a preferred embodiment, the initial pH of the buffered hydrogel is 7, but according to this embodiment can range from being mildly acidic (pH of 5) to fully basic (pH of 12). In a preferred embodiment, the buffering compound used in the electrode with the hydrogel is magnesium acetate, although it will be understood that other buffering compounds can be selected.

It should be noted that there are other electrochemically-based treatment systems that employ a third electrode, often referred to as a reference electrode in addition to the working electrode and a counter electrode. One example is described in U.S. Patent Application 62/962,524, filed Jan. 17, 2020, and entitled: Galvanostatic Method of Microbe Removal From Surgically Implanted Orthopedic Devices, the entire contents of which are incorporated herein by reference. For these treatments, it is important that the reference electrode be placed in a consistent anatomical location from patient to patient. A reference electrode is an electrode which has a stable and well-known electrode potential. This latter electrode can be adhered to the skin of the patient. In potentiostatic circuits specifically, reference electrode potentials are used as an actual reference to compare with the applied working electrode potential. In other words, if the user applies a certain amount of voltage to the working electrode with respect to the reference electrode, the voltage will remain electrochemically stable because the reference potential is stable. A fundamental behavior in potentiostatic systems, such as CVCES treatment systems, is that the resistance between the working electrode (the implant) and the reference electrode causes a certain amount of current to be driven as a result of the applied voltage. However, the current that the reference electrode dictates is driven between the working electrode and the counter electrode.

The resistance between a surgical implant and the reference electrode can change from patient to patient due to several factors, including skin condition and tissue composition among others. This resistance can cause inconsistencies in treatment from patient to patient because the current is what creates the therapeutic chemical reaction. One way to optimize the consistency between the reference to working resistance is to insure that the reference electrode is always placed in a consistent anatomical location with respect to the metal implant. According to at least one embodiment, the inventive electrode can contain at least one feature that is configured to align with anatomical landmarks of the body of the patient, such as the patella. The at least one alignment feature enables consistent placement zones for the reference electrode, thereby leading to provide more consistent treatment in terms of current draw.

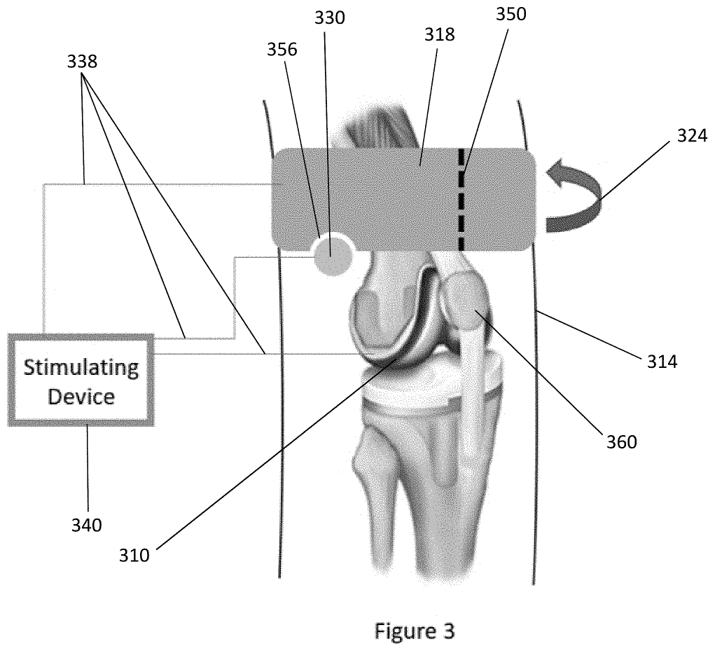

FIG. 3 shows an annotated view of an idealized anatomical knee image, including an electrode made in accordance with the present invention for use in an electrochemical metal implant treatment system having three (3) separate electrodes; namely a working electrode (the metal implant 310), a counter electrode 318 and a reference electrode 330. In this figure, the depicted lines 314 represent the skin boundaries of the leg of a patient with the metal implant 310 being a knee replacement having tibial and femoral components. The rectangle extending over the upper portion of the knee 300 represents the counter electrode 318, which similar to electrode 200, FIG. 2, is sized and configured to be circumferentially wrapped about the entirety of the leg, as shown by arrow 324. The circular region shown represents the reference electrode 330, with electrical connections represented as lines 338 leading from the three (3) depicted electrodes 310, 318, 330 back to a stimulating device 340, such as a potentiostat.

According to this embodiment, the illustrated circumferential counter electrode 318 has at least two (2) features that help reference electrode consistency. First, a dotted line 350 or similarly denoted section provided on the exterior of the counter electrode 318 is an alignment aid that enables the physician to align the center of the counter electrode 318 repeatedly in line with an anatomical landmark (e.g., the patella 360) of the patient. The patella 360 is an easy to identify anatomical landmark that provides a good reference for the physician though other similar landmarks can be utilized, depending, for example, on the surgical site and procedure. Second and according to this specific embodiment, the counter electrode 318 is provided with an identifiable placement zone or area 356 such as a cut-out area or other easily identifiable feature that provides a consistent spot for the reference electrode 330 to adhere to the skin of the patient. This placement zone 356 will always be the same distance from the patellar alignment. In a preferred embodiment, the placement zone 356 will be land on the medial side of the leg of the patient.

The herein described electrode may contain additional features to increase efficiency on the device side of the electrical stimulation. Typically, electrodes for use in implant treatment systems are electrically attached via a single point of contact to the lead wire extending to the stimulating device. With an electrode as large as the herein described wrappable circumferential design, a single point of contact would require the stimulating device to provide higher than typical electrical potentials to provide an adequate treatment. This behavior results from the material that the electrode is made from. In direct current (DC) applications, the conductive surface that interfaces with the hydrogel needs to be conductive, yet inert, so to not corrode as a part of the resulting anodic reaction. Because of this need, a typical material of choice for the inert and anodic conductive layer is a carbonized rubber or carbon film. Carbon is an inert and conductive material, but is comparably less conductive than traditional conductors, such as copper.

In order for the electrical current from the stimulating device to spread over a larger surface area based on the larger wrappably sized electrode, as compared to the typical patch electrode 100, FIG. 1, the potentiostat or other suitable stimulating device must compensate for the losses that come from less conductive carbon by increasing the voltage to the counter electrode. Limitations in the stimulation device may not be able to provide the elevated voltage potentials required by the counter electrode in some situations, which can starve the counter electrode of electrical potential, thereby limiting the current and treatment to the implant. Therefore and to keep the counter electrode voltage requirement as low as possible, the inventive electrode preferably includes a conductive layer behind the inert conductive carbon surface layer of the electrode, which spreads the point of contact over a considerably larger area. In this specifically described embodiment, the conductive layer shown is made from a copper mesh, though this layer can alternatively be made from a solid sheet of a suitable electrically conductive material.

With reference to FIG. 4, a sectioned view of the various electrode layers of a portion of an electrode 400 made in accordance with an exemplary embodiment of the invention is presented. As noted, only a portion of the electrode 400 is shown in this view, which is preferably sized and has sufficient flexibility to be wrapped entirely or at least over a majority (greater than 50 percent) about the limb of a patient in proximity to an implanted orthopedic device. According to this embodiment, the bottommost layer of the electrode 400 represents the buffered hydrogel layer 406, the adjacent layer (second from the bottom layer) represents the anodic and chemically inert conductive layer 412, the second layer from the top represents the conductive layer 416, and the top or uppermost layer of the electrode 400 represents a fabric adhesive layer 422. All of the resident layers 406, 412, 416 are preferably coextensive with the fabric adhesive layer 422.

As shown in this embodiment, an electrical connection such as provided by a lead wire 404 extending from a stimulating device (not shown in this view) attaches through the back of the fabric adhesive layer 422 to electrically connect to the conductive layer 416, which as noted previously is preferably made from copper. The conductive layer 416 easily conducts the electrical potential through its surface to electrify the anodic conductive layer 412, which according to a preferred embodiment is made from a chemically inert material such as carbon. Therefore, no spot on the anodic surface layer 412 is a large distance away from where the electricity transfers from the conductive (copper) layer 416 to the anodic and inert conductive layer 412. This configuration reduces electrical losses in the anodic conductive layer 412 and lowers the electrical potential demand of the counter electrode 400 to provide the required current to the metal implant (not shown in this view). In a preferred embodiment, the conductive layer 416 has a thickness of 0.01-0.02 inches to remain flexible, but the thickness of this layer can range between 0.001 to 0.1 inches. The fabric adhesive layer 422 includes an adhesive backing that adheres through the openings in the conductive layer 416 to the inert conductive layer 412 in order to lock the configuration in place. The lead wire 404 as shown is attached to the exterior of the flexible covering 422, wherein the configuration can alternatively be sealed between the conductive layer 416 and the covering 422.

In use, the electrode is wrapped about the limb of a patient (not shown) proximate the implanted orthopedic device. A stimulating device of the treatment system provides the required current and via the coupling of the coextensive conductive layer 416, electrifies the inert conductive layer 412, the latter forming the anode of the electrochemical cell created between the implant (cathode) and the electrode with the patient providing the electrolytic solution for the resulting oxidation and reduction reactions to remove biofilms from a surface of the implanted orthopedic device via the conductive interface provided by the hydrogel layer.

### PARTS LIST FOR FIGS. 1-4

100 electrode, patch (skin)
104 metal implant
110 stimulating device/potentiostat
114 connections, electrical
116 skin, patient
120 shading/flesh
124 stars representing anodic current flow
128 stars representing cathodic current flow
200 electrode
300 knee
310 metal implant
314 skin boundaries, leg
318 counter electrode
324 circumferential orientation, counter electrode
330 reference electrode
338 electrical connections
350 alignment feature
356 placement zone or area, reference electrode
360 patella
400 electrode
404 lead wire
406 buffered hydrogel layer
412 inert conductive layer
416 conductive layer
422 fabric adhesive layer

It will be understood that modifications and variations are possible in accordance with the following claims:

1-26. (canceled)

27. A wrappable electrode configured for use in an electrochemical-based system for removing biofilm from a metal surgically implanted device, the system comprising the metal surgically implanted device as a working electrode, the wrappable electrode as a counter electrode and a reference electrode, the wrappable electrode comprising:
a flexible covering;
a lead wire configured for connecting the wrappable electrode to a stimulating device of the system that produces a cathodic voltage;
an adhesive layer to enable the wrappable electrode to be attached to the skin of a patient, the adhesive layer containing a chemically buffered hydrogel used to neutralize acidic chemical byproducts of an anodic reaction that occurs when the cathodic voltage is produced;
an inert conductive layer to which the lead wire is electrically coupled; and
a conductive layer separate from the inert conductive layer and disposed between the inert conductive layer and the flexible covering in which the inert conductive layer is sized in conformance with the separate conductive layer and the flexible covering so as to isolate the separate conductive layer and the lead wire from the chemically buffered hydrogel, wherein the wrappable electrode is sized and configured to be wrapped circumferentially about at least a portion of a limb of a patient in proximity to the metal surgically implanted device so as to evenly and uniformly distribute treatment in the form of current to a surface of the metal surgically implanted device and further to dilute the acidic chemical byproducts.

28. The wrappable electrode according to claim 27, wherein the wrappable electrode is sized and configured to be wrapped at least over 50 percent of the circumference of the limb.

29. The wrappable electrode according to claim 28, wherein the wrappable electrode is sized and configured to be entirely wrapped about the circumference of the limb.

30. The wrappable electrode according to claim 27, in which the conductive layer separate from the inert conductive layer is made from copper.

31. The wrappable electrode according to claim 27, further comprising at least one feature disposed on the flexible covering configured to align the wrappable electrode relative to a landmark feature of a patient.

32. The wrappable electrode according to claim 27, further comprising at least one feature for positioning the reference electrode.

33. The wrappable electrode according to claim 32, in which the at least one feature for positioning the reference electrode comprises a cut out area formed on the wrappable electrode.

34. A system for treatment of a metallic implanted device, the system comprising:
a stimulating device capable of producing a cathodic voltage;
a reference electrode;
a wrappable electrode attached to the skin of a patient in proximity to the metallic implanted device, each of the metallic implanted device, the reference electrode and the wrappable electrode being electrically coupled to the stimulating device in which the metallic implanted device is a working electrode and the wrappable electrode is a counter electrode of a formed electrochemical cell, and in which the wrappable electrode is sized and configured to be circumferentially wrapped about at least a portion of the circumference of a limb of the patient in order to evenly and uniformly distribute treatment to a surface of the metallic implanted device, the wrappable electrode comprising:
a flexible covering;
a lead wire for electrically connecting the wrappable electrode to the stimulating device;
an adhesive layer to enable the wrappable electrode to be attached to the skin of a patient, the adhesive layer having a chemically buffered hydrogel used to neutralize acidic chemical by-products of an anodic reaction that occurs when the cathodic voltage is produced by the stimulating device;
an inert conductive layer to which the lead wire is attached; and
a conductive layer separate from the inert conductive layer and disposed between the inert conductive layer and the flexible covering to which the lead wire is attached, the inert conductive layer being sized and configured in order to shield the separate conductive layer and the lead wire from the chemically buffered hydrogel.

35. The system according to claim 34, in which the conductive layer separate from the inert conductive layer is made from copper.

36. The system according to claim 34, wherein the wrappable electrode is sized and configured to be wrapped at least 50 percent about the circumference of the limb.

37. The system according to claim 34, wherein the wrappable electrode is sized and configured to be wrapped entirely about the circumference of the limb.

38. The system according to claim 34, wherein the wrappable electrode further comprises at least one feature disposed on the flexible covering configured to align the wrappable electrode relative to the patient.

39. The system according to claim 34, wherein the the wrappable electrode further comprises at least one feature for positioning the reference electrode.

40. The system according to claim 39, in which the at least one feature for positioning the reference electrode comprises at least one cut-out area.

41. The system according to claim 34, in which the inert conductive layer is made from carbon.

42. The system according to claim 34, in which the conductive layer separate from the inert conductive layer is a mesh.

43. A method for manufacturing an electrode for an electrochemically-based system configured to remove biofilm from a metal surgically implanted device in which the metal surgically implanted device is a working electrode and the manufactured electrode is a counter electrode, the method comprising:
providing a flexible covering sized to cover at least a circumferential portion of a limb of a patient having the metal surgically implanted device;
providing a lead wire couplable to a stimulating device of the system capable of delivering a cathodic voltage;
providing an adhesive layer enabling the electrode to be attached to the skin of the patient, including the step of providing a chemically buffered hydrogel in the adhesive layer for neutralizing acidic byproducts of an anodic reaction that occurs when the cathodic voltage is produced;
providing an inert conductive layer in relation to the adhesive layer and the chemically buffered hydrogel;
providing a separate conductive layer between the flexible covering and the inert conductive layer and attaching the lead wire to the separate conductive layer, and
sizing the inert conductive layer so as to isolate the lead wire from the chemically buffered hydrogel.

44. The method according to claim 43, further comprising:
providing at least one feature on the flexible covering for positioning a reference electrode of the electrochemically-based system.

45. The method according to claim 44, wherein the at least one feature for positioning the reference electrode is a cut-out area.

46. The method according to claim 43, further comprising:
providing at least one feature on the flexible covering configured to align the wrappable electrode relative to the patient.
